(19) United States
(12) Patent Application Publication
(10) Pub. No.: US 2025/0186750 A1
(43) Pub. Date: Jun. 12, 2025

(54) Adjunct Non-Pneumatic Operative Field for Robotic/Endoscopic Surgery

(71) Applicant: Fadi N. Bashour, Westlake, OH (US)

(72) Inventor: Fadi N. Bashour, Westlake, OH (US)

(21) Appl. No.: 18/531,813

(22) Filed: Dec. 7, 2023

(51) Int. Cl. A61M 29/00

(52) U.S. Cl. CPC A61M 29/00

(57) ABSTRACT

An adjunct non-pneumatic operative field device having a pair of selectively lockable, slidingly engaged nested tubes. The pair of tubes consist of an inner tube and an over tube. Each tube has a proximal end and a distal end. The distal ends could be tapered. In addition, this adjunct non-pneumatic operative field device has a lock. The adjunct non-pneumatic operative field device has two interchangeable configurations: a home position and an active position. In the home position the two distal ends are either coterminous or adjacent to each other. Alternatively, in the active position, the two distal ends are spaced apart by a user. And the active position engages the lock, which suppresses relative movement between the selectively lockable, slidingly engaged nested tubes. The active position also consists of expansion of the over tube to create an operative secure field.

### CROSS-REFERENCE TO RELATED APPLICATIONS

Not applicable.

### STATEMENT REGARDING FEDERALLY SPONSORED RESEARCH AND DEVELOPMENT

Not applicable.

### THE NAMES OF THE PARTIES TO A JOINT RESEARCH AGREEMENT

Not applicable.

### STATEMENT REGARDING PRIOR DISCLOSURES BY THE INVENTOR OR A JOINT INVENTOR

Not applicable.

### BACKGROUND OF THE INVENTION

The present disclosure relates to devices used in endoscopic and robotic surgeries. More particularly, it pertains to a device creating an adjunct non-pneumatic operative field for these surgeries.

During endoscopic and robotic surgeries, physicians and surgeons can remove polyps, tumors, and other growths. For example, in colorectal cancer, without removal of these growths, patients diagnosed with or predisposed to this type of cancer may have increased mortality and morbidity.

However, surgery requires certain conditions for an optimal outcome. One such condition is necessary operative space, which creates access to perform surgery.

Alternatively, potential complications from incomplete space include, among other things, inadequate utilization of surgical instruments. This lack of operative space increases the risk for eliminating healthy tissue inadvertently due to healthy tissue not being visually apparent or adequately distended. Furthermore, lack of operative space near the procedure inhibits the skilled artisan's ability to detect additional polyps, tumors, or other growths that might be near the surgery's vicinity. Lack of detection could leave growths in the patient, thereby allowing progression to higher stages of cancer.

One potential solution for increased space is insufflation (i.e., introduction of air), which also provides increased visualization. The increased pressure in the alimentary canal distends it, providing more visibility in the canal. However, the air utilized in insufflation would not create an operatively secure area for surgery.

Recognizing the shortfall of insufflation, others have created devices to form operative space. One such attempt—disclosed in U.S. Pat. No. 11,553,830—is an alimentary device with a balloon stationed further into the alimentary canal beyond the alimentary device. However, even with the increased operative space, this balloon might not provide an operatively secure field for surgical instruments. This situation would be prevalent in surgery where surgical instruments are coming near the balloon, which threatens puncture of the balloon and subsequent loss of the field.

Therefore, to obtain an operatively secure field during surgery, a device is needed to create a fixed distension in the alimentary canal. The fixed distension could have multiple settings for distensions. The fixed distensions would provide a broader—and secure—area for physicians, surgeons, and skilled artisans to work within. This improvement would create a distended space to work within, in addition to an increased field of view without the accompanying risk of lacerating a balloon responsible for the distension.

### BRIEF SUMMARY OF THE INVENTION

Provided in this disclosure is an adjunct non-pneumatic operative field device for robotic and endoscopic surgeries. This field device provides a more fixed field during surgery. In addition, this device provides a variable operative field for robotic/endoscopic surgery.

One aspect of this disclosure is a pair of selectively lockable, slidingly engaged nested tubes. The pair of nested tubes consist of an inner tube and an over tube. The inner tube has a proximal end and a distal end. In an exemplary embodiment, the distal end of the inner tube could be tapered and could have a basket-shaped end (i.e., basket). The basket-shaped end could be made of a plastic polymer with a memory shape. In addition, the basket-shaped end could be of variable length. Regarding the inner tube's tapering, the tapering would be at any angle less than ninety degrees, creating a beveled surface. The over tube has a proximal end and a distal end. In another exemplary embodiment, the distal end of the over tube could be tapered. Similarly, the tapering would be at any angle less than ninety degrees, which would create a beveled surface. These tubes could be fabricated from plastic, polymeric material, any other material used by the skilled artisan for tubes used in the alimentary canal, or any combination thereof. In addition, the tubes may create a basket-like operative field that can vary in size depending on the position of the inner tube, the over tube, or both.

According to another aspect of the disclosure, the adjunct non-pneumatic operative field device has a lock. In an exemplary embodiment, this lock would be mechanical and have a control whereby a skilled artisan could selectively lock the nested tubes in a certain configuration. The control could be mechanical, electronic, or any other control means used by the skilled artisan.

An exemplary embodiment of a mechanical lock is one utilizing a mechanical tab or a plurality of mechanical tabs to inhibit movement between the nested tubes. The mechanical tab or plurality of mechanical tabs would be on at least one side of the over tube or the inner tube to create resistance sufficient to inhibit relative movement between the nested tubes. In addition, the lock could use another type of mechanical lock, an electronic lock, or any other type of lock used by the skilled artisan to suppress relative movement between nested tubes.

According to a further aspect of the present disclosure, the pair of selectively lockable, slidingly engaged nested tubes can transition between a home position and an active position. In the home position, the two distal ends would be coterminous or adjacent. Regardless, the proximity of the two distal ends, provided by being coterminous or adjacent, allows the skilled artisan to place the adjunct non-pneumatic operative field device more easily into a patient's alimentary canal.

Alternatively, once placed into the alimentary canal, a skilled artisan can utilize the adjunct non-pneumatic operative field device for surgery, including robotic and endoscopic procedures. Part of this utilization is achieved by the active position of the adjunct non-pneumatic operative field device. In the active position, the two distal ends are spaced apart from their past proximity.

According to an additional aspect of the disclosure, the active position also engages the lock, which engages to suppress relative movement between the selectively lockable, slidingly engaged nested tubes. This suppression of relative movement allows a physician, surgeon, or a skilled artisan to create and maintain an operative secure field for surgery.

According to another aspect of the present disclosure, the pair of selectively lockable, slidingly engaged nested tubes engage in the active position, expanding the over tube at its distal end to create an operative secure field. Such a field empowers a skilled artisan to use the adjunct non-pneumatic operative field device in conjunction with surgical instruments in surgery.

According to yet another aspect of the present disclosure, the expansion of the over tube at its distal end occurs through mechanical arms that push the over tube from the home position to the active position, distending the over tube closer to the interior walls of the alimentary canal. However, the skilled artisan would understand the over tube could be expanded by other mechanical implements, such as tabs, probes, slats, or extensions sufficient for the over tube to selectively engage the interior walls of the alimentary canal.

Also provided in this disclosure is a method of using an adjunct non-pneumatic operative field device for surgeries. The method includes the steps of: (i) inserting the operative secure field device, while in the home position, into a patient's alimentary canal, (ii) sliding the adjunct non-pneumatic operative field device inside of the patient to a desired position within the alimentary canal, and (iii) engaging the adjunct non-pneumatic operative field device in an active position.

Also provided in this disclosure is an alternative method of using an adjunct non-pneumatic operative field device for surgeries. The method includes the steps of: (i) inserting the operative secure field device, while in the home position, into a patient's alimentary canal, (ii) engaging the adjunct non-pneumatic operative field device in an active position, and (iii) sliding the adjunct non-pneumatic operative field device further inside the patient to a desired position within the alimentary canal.

However, the skilled artisan would understand that additional obvious steps could be incorporated, such as placing the device into the desired portion of the alimentary canal before engaging the operative secure field device, without departing from the disclosed methods.

### BRIEF DESCRIPTION OF THE SEVERAL VIEWS OF THE DRAWINGS

The accompanying drawings in the following description illustrate various exemplary embodiments of the present disclosure. It is understood that a person of ordinary skill in the art may derive other embodiments from these drawings, which fall within the scope of the disclosure set forth herein.

FIG. 1 illustrates a front view of an embodiment of the operative secure field device.

FIG. 2 illustrates a side view of an embodiment of the operative secure field device.

FIG. 3 illustrates another side phantom view of an embodiment of the operative secure field device.

FIG. 4 illustrates another side phantom view of an embodiment of the operative secure field device.

FIG. 5 illustrates another side view of an embodiment of an adjunct non-pneumatic operative field device inside an alimentary canal.

FIG. 6 illustrates another side view of an embodiment of an adjunct non-pneumatic operative field device inside an alimentary canal.

FIG. 7 illustrates a rear isometric phantom view of an embodiment of the operative secure field device.

FIG. 8 illustrates another rear isometric phantom view of an embodiment of an operative secure field device.

FIG. 9 illustrates another rear isometric view of an embodiment of an operative secure field device.

The drawings will be described in greater detail below.

### DETAILED DESCRIPTION OF THE INVENTION

Reference is now made to the drawings wherein the showings are for purposes of illustrating aspects of the device only and not for purposes of limiting the same, and wherein like reference numerals are understood to refer to like components.

The present disclosure is directed to an adjunct non-pneumatic operative field device (10) able to create an operatively secure field and its methods of use. The adjunct non-pneumatic operative field device (10) can engage sections of the alimentary canal (90) within both the upper and lower gastrointestinal tract. Accordingly, the adjunct non-pneumatic operative field device (10) is useful for endoscopic and robotic surgeries. During these procedures, a skilled artisan can insert the adjunct non-pneumatic operative field device (10) into a patient's lower gastrointestinal tract, such as the colon or large intestine. Alternatively, a skilled artisan may place the adjunct non-pneumatic operative field device (10) into a patient's upper gastrointestinal tract, such as the esophagus, stomach, or duodenum.

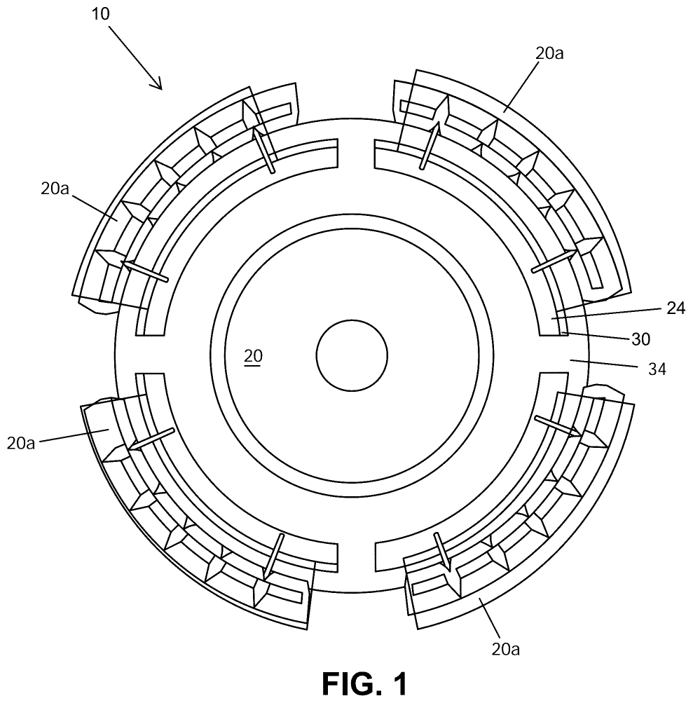

FIG. 1 shows a front view of the adjunct non-pneumatic operative field device (10) comprising a pair of selectively lockable, slidingly engaged nested tubes (20, 30). The inner tube (20) has a distal end (24). The over tuber (30) has a distal end (34). The inner tube (20) is nested in the over tube (30). The inner tube (20) also comprises a basket (20a) as a part of its distal end (24). The distal ends (24, 34) of the pair of selectively lockable, slidingly engaged nested tubes (20, 30) are in adjacency.

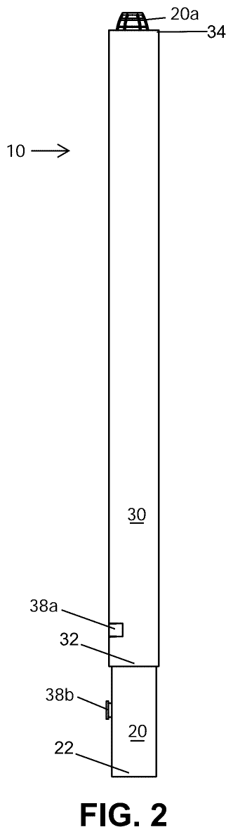

FIG. 2 depicts a side view of the adjunct non-pneumatic operative field device (10), which comprises a pair of selectively lockable, slidingly engaged nested tubes (20, 30). The two selectively lockable, slidingly engaged tubes (20, 30) comprise an inner tube (20) and an over tube (30), which are in adjacency. The inner tube (20) could be circular, cylindrical, or any other shape used by the skilled artisan for use in the alimentary canal (90). The over tube (30) could be circular, cylindrical, or any other shape used by the skilled artisan for surgical use in the alimentary canal (90).

FIG. 2 also shows the inner tube (20) with a proximal end (22) and a distal end (24). The over tube (30) has a proximal end (32) and a distal end (34). The inner tube (20), comprising the basket (20a), is partially nested in the over tube (30).

FIG. 2 additionally shows the adjunct non-pneumatic operative field device (10) with a lock (38). The lock (38) consists of a path (38a) on the over tube (30) and a tab (38b). A user can urge the tab (38b) towards the distal end of the over tube (30) along the path (38a). Once lateral movement is achievable on the path (38a), the tab (38b) resists relative movement on the path (38a). Once laterally moved, the lock (38) is selectively engaged, thereby allowing the over tube (30) to resist relative movement with the inner tube (20) following engagement of the nested tubes (20, 30).

FIG. 2 also shows the pair of selectively lockable, slidingly engaged nested tubes (20, 30) have a home position (40). In the home position (40), the lock (38) is not selectively engaged, which does not suppress relative movement of the pair of selectively lockable, slidingly engaged nested tubes (20, 30).

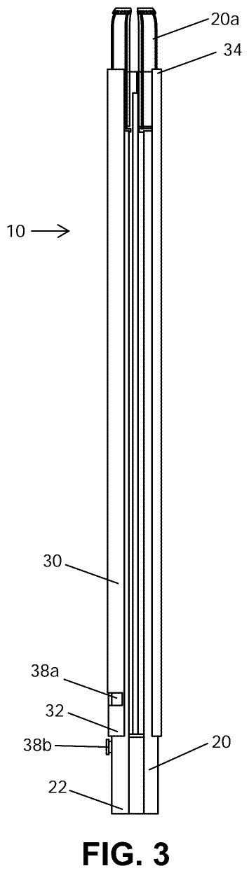

FIG. 3 shows the adjunct non-pneumatic operative field device (10) transitioning between the home position (40) and the active position (50). Relative to FIG. 2, the tab (38b) has been urged closer to the proximal end (32) of the over tube (30). Accordingly, the basket (20a) of the inner tube (20) has been extended beyond its home position (40), as originally depicted in FIG. 2.

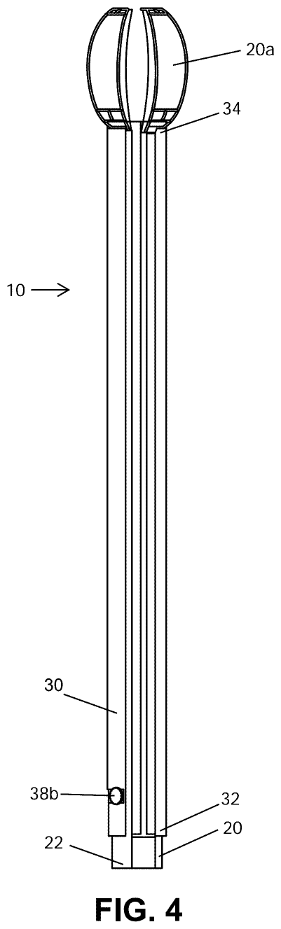

FIG. 4 shows the adjunct non-pneumatic operative field device (10) in the active position (50). Specifically, FIG. 4 shows the pair of selectively lockable, slidingly engaged nested tubes (20, 30) in their active position (50). Furthermore, the basket (20a) of the inner tube (20) has been distally extended. In combination with FIG. 3, a skilled artisan would understand the adjunct non-pneumatic operative field device (10) can reversibly interchange between the two positions (40, 50) depending on a patient's need or the phase of the surgery.

As FIG. 4 illustrates, the adjunct non-pneumatic operative field device (10) has the active position (50) wherein the two distal ends (24, 34) are spaced apart further than the adjacency in the home position (40). In addition, the active position (50) utilizes the lock (38) to selectively engage the lock (38), which suppresses relative movement of the pair of selectively lockable, slidingly engaged nested tubes (20, 30). Lastly, the movement from the home position (40) to the active position (50) induces the inner tube (20) to expand its basket (20a), creating the operatively secure field.

Comparing FIGS. 2, 3, and 4, in FIG. 2, the adjunct non-pneumatic operative field device (10) is in the home position (40), wherein the two distal ends (24, 34) are in adjacency. This adjacency inhibits the basket (20a) from expanding; in contrast, the active position (50) helps create the operatively secure field beyond the distal end (34) of the over tube (30).

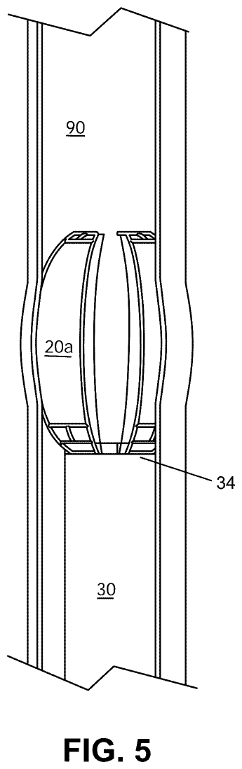

FIG. 5 depicts the adjunct non-pneumatic operative field device (10) inside an alimentary canal (90). The adjunct non-pneumatic operative field device (10) is in the active position (50), wherein the two distal ends (24, 34) are spaced apart further than the adjacency in the home position (40). The active position (50) uses the inner tube (20)—specifically its distal end (24) and the basket (20a)—to expand, creating the operative secure field past the distal end (34) of the over tube (30).

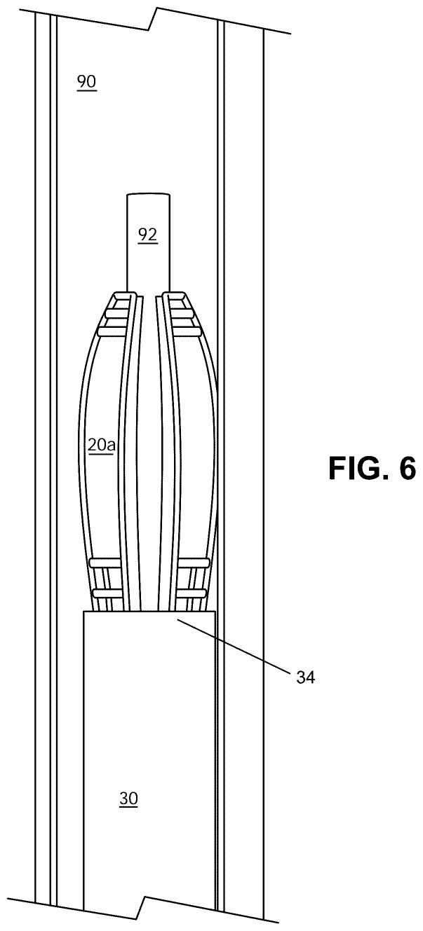

FIG. 6 depicts the adjunct non-pneumatic operative field device (10) inside an alimentary canal (90). The adjunct non-pneumatic operative field device (10) is in the active position (50), the two distal ends (24, 34) are spaced apart further than the adjacency in the home position (40). In addition, the active position (50) shows the lock (38) that has been selectively engaged, which suppresses relative movement of the pair of selectively lockable, slidingly engaged nested tubes (20, 30). The active position (50) uses the inner tube (20)—specifically its distal end (24) and the basket (20a)—to expand, creating the operative secure field. The operative secure field can be variable lengths depending on the length the inner tube (20) is urged past the distal end (34) of the over tube (30). Accordingly, a surgical implement (92) can nest in the inner tube (20) and its basket (20a). The skilled artisan would understand the surgical implement (92) to be any surgical implement (92) used in surgery, including an endoscope for endoscopic or robotic surgery.

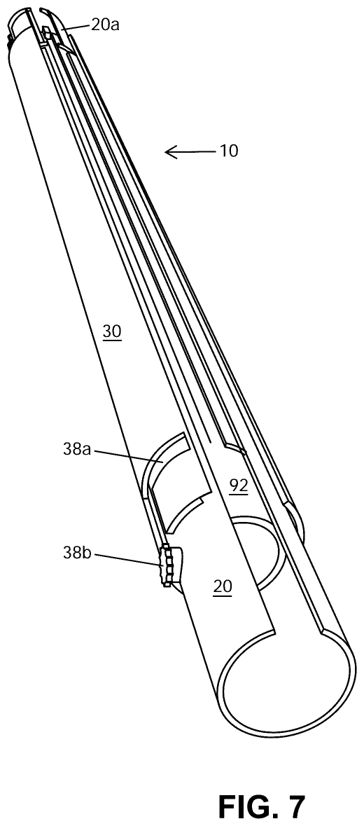

FIG. 7 shows the adjunct non-pneumatic operative field device (10) in the home position (40). The lock (38) has not been selectively engaged, which does not suppress relative movement of the pair of selectively lockable, slidingly engaged nested tubes (20, 30). In addition, the surgical implement (92) is inside the inner tube (20).

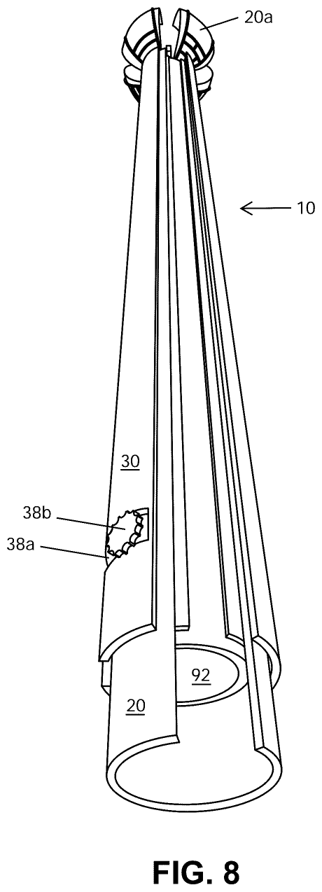

FIG. 8 shows the adjunct non-pneumatic operative field device (10) in the active position (50). The lock (38) has been selectively engaged, where the tab (38b) has been moved laterally once reaching the most distal portion of the path (38a) which suppresses relative movement of the pair of selectively lockable, slidingly engaged nested tubes (20, 30). In addition, the basket (20a) has been distally extended.

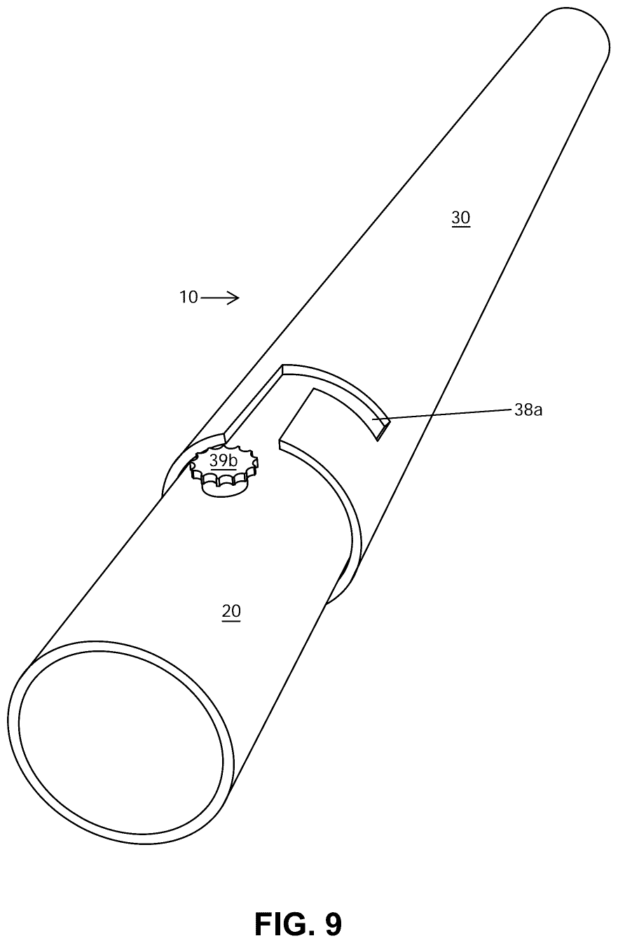

After the sequence from FIG. 7 to FIG. 8, FIG. 9 shows the adjunct non-pneumatic operative field device (10) can transition back to the home position (40) from the active position (50). The lock (38) has been selectively disengaged, wherein the tab (38b) is not in the path (38a), which does not suppress relative movement of the pair of selectively lockable, slidingly engaged nested tubes (20, 30).

Also provided in this disclosure is a method of using an adjunct non-pneumatic operative field device (10) for surgeries, such as endoscopic or robotic surgeries. The method includes the steps of: (i) inserting the adjunct non-pneumatic operative field device (10) in the home position (40) into the alimentary canal (90) and (ii) sliding the adjunct non-pneumatic operative field device (10) inside of the patient to a desired position within the alimentary canal (90). Lastly, referring to FIG. 5, the step of (iii) engaging the adjunct non-pneumatic operative field device (10) in an active position (50) to create the operatively secure field.

In addition, according to another aspect of this disclosure, a similar method of using the adjunct non-pneumatic operative field device (10) for surgeries. This method includes the steps of: (i) inserting the adjunct non-pneumatic operative field device (10) in the home position (40) into the alimentary canal (90), (ii) engaging the adjunct non-pneumatic operative field device (10) in an active position (50) to create the operatively secure field, and (iii) sliding the adjunct non-pneumatic operative field device (10) inside of the patient to a desired position within the alimentary canal (90).

While the apparatus, system, and method have been described with reference to various embodiments, those skilled in the art will understand that various changes may be made, and equivalents may be substituted for elements thereof, without departing from the scope and essence of the disclosure. In addition, many modifications may be made to adapt a particular situation or material in accordance with the teachings of the disclosure without departing from the essential scope thereof.

Therefore, it is intended that the disclosure is not limited to the embodiments disclosed, but that the disclosure will include all embodiments falling within the scope of the appended claims. In this application all units are in the metric system and all amounts and percentages are by weight, unless otherwise expressly indicated. Also, all citations referred to herein are expressly incorporated herein by reference.

1. An adjunct non-pneumatic operative field device (10) comprising:
a pair of selectively lockable, slidingly engaged nested tubes (20, 30) comprising:
i. an inner tube (20) having a proximal end (22) and a distal end (24),
ii. an over tube (30) having a proximal end (32) and a distal end (34), and
iii. a lock (38),
wherein the pair of selectively lockable, slidingly engaged nested tubes have a home position (40) and an active position (50),
wherein in the home position the two distal ends (24, 34) are in adjacency,
wherein in the active position,
i. the two distal ends are spaced apart by a user,
ii. the lock is selectively engaged to suppress relative movement between the pair of selectively lockable, slidingly engaged nested tubes, and
iii. the over tube at its distal end expands to create an operative secure field.

2. The adjunct non-pneumatic operative field device of claim 1, wherein the distal end of the inner tube is tapered.

3. The adjunct non-pneumatic operative field device of claim 1, wherein the distal end of the over tube is tapered.

4. The adjunct non-pneumatic operative field device of claim 1, wherein the over tube is made of a polymeric material.

5. The adjunct non-pneumatic operative field device of claim 1, wherein the over tube is made of rubber.

6. The adjunct non-pneumatic operative field device of claim 1, wherein the over tube at its distal end expands to engage an interior wall of an alimentary canal.

7. The adjunct non-pneumatic operative field device of claim 1, wherein the over tube at its distal end expands to engage an entire circumference of an interior wall of an alimentary canal.

8. An adjunct non-pneumatic operative field device (10) comprising:
a pair of selectively lockable, slidingly engaged nested tubes (20, 30) comprising:
i. an inner tube (20) having a proximal end (22) and a distal end (24),
ii. an over tube (30) having a proximal end (32) and a distal end (34), and
iii. a lock (38),
wherein the pair of selectively lockable, slidingly engaged nested tubes have a home position (40) and an active position (50),
wherein in the home position the two distal ends (24, 34) are coterminous,
wherein in the active position,
i. the two distal ends are spaced apart by a user,
ii. the lock is selectively engaged to suppress relative movement between the pair of selectively lockable, slidingly engaged nested tubes, and
iii. the over tube at its distal end expands to create an operative secure field.

9. The adjunct non-pneumatic operative field device of claim 8, wherein the distal end of the inner tube is tapered.

10. The adjunct non-pneumatic operative field device of claim 8, wherein the distal end of the over tube is tapered.

11. The adjunct non-pneumatic operative field device of claim 8, wherein the over tube is made of a polymeric material.

12. The adjunct non-pneumatic operative field device of claim 8, wherein the over tube is made of a polymeric material.

13. The adjunct non-pneumatic operative field device of claim 8, wherein the over tube is made of rubber.

14. The adjunct non-pneumatic operative field device of claim 8, wherein the over tube at its distal end expands to engage an interior wall of an alimentary canal.

15. The adjunct non-pneumatic operative field device of claim 8, wherein the over tube at its distal end expands to engage an entire circumference of an interior wall of an alimentary canal.

16. A method of using the adjunct non-pneumatic operative field device of claim 1,
wherein the method comprises the following steps:
a. inserting the adjunct non-pneumatic operative field device in the home position into a patient's alimentary canal,
b. sliding the adjunct non-pneumatic operative field device inside of the patient to a desired position within the alimentary canal, and
c. engaging the adjunct non-pneumatic operative field device in the active position.

17. A method of using the adjunct non-pneumatic operative field device of claim 1,
wherein the method comprises the following steps:
a. inserting the adjunct non-pneumatic operative field device in the home position into a patient's alimentary canal,
b. engaging the adjunct non-pneumatic operative field device in the active position, and
c. sliding the adjunct non-pneumatic operative field device inside of the patient to a desired position within the alimentary canal.

18. A method of using the adjunct non-pneumatic operative field device of claim 8,
wherein the method comprises the following steps:
a. inserting the adjunct non-pneumatic operative field device in the home position into a patient's alimentary canal,
b. sliding the adjunct non-pneumatic operative field device inside of the patient to a desired position within the alimentary canal, and
c. engaging the adjunct non-pneumatic operative field device in the active position.

19. A method of using the adjunct non-pneumatic operative field device of claim 8,
wherein the method comprises the following steps:
a. inserting the adjunct non-pneumatic operative field device in the home position into a patient's alimentary canal,
b. engaging the adjunct non-pneumatic operative field device in the active position, and
c. sliding the adjunct non-pneumatic operative field device inside of the patient to a desired position within the alimentary canal.

20. An adjunct non-pneumatic operative field device (10) comprising:
a pair of selectively lockable, slidingly engaged nested tubes (20, 30) comprising:
i. an inner tube (20) having a basket (20a), a proximal end (22), a distal end (24),
ii. an over tube (30) having a proximal end (32) and a distal end (34),
iii. a lock (38) comprising at least one tab (38b) and a path (38a), and
iv. at least one arm (39) able to expand the inner tube,
wherein the pair of selectively lockable, slidingly engaged nested tubes have a home position (40) and an active position (50),
wherein in the home position the two distal ends (24, 34) are coterminous,
wherein in the active position,
i. the two distal ends are spaced apart,
ii. the lock is selectively engaged to suppress relative movement between the pair of selectively lockable, slidingly engaged nested tubes, and
iii. at least one arm of the adjunct non-pneumatic operative field device expands the distal end of the inner tube to create an operative secure field.
